(19) United States
(12) Patent Application Publication
(10) Pub. No.: US 2003/0206893 A1
(43) Pub. Date: Nov. 6, 2003

(54) Cell proliferating agents

(76) Inventor: Sohail Malik, Roswell, GA (US)

Correspondence Address: PAULEY PETERSEN KINNE & ERICKSON, 2800 WEST HIGGINS ROAD, SUITE 365, HOFFMAN ESTATES, IL, 60195, US

(21) Appl. No.: 10/140,270

(22) Filed: May 6, 2002

(51) Int. Cl. A61K 38/43; A61K 31/19; A01N 63/00; A01N 25/00

(52) U.S. Cl. 424/94.1; 504/118; 504/144; 514/573

(57) ABSTRACT

Pharmaceutical compositions and methods of using the same are provided utilizing effective amounts of one or more plant growth factors, gibberellic acid, kinetin, zeatin and jasmonic acid to increase cell proliferation in various tissues and cell lines. As examples, the compositions and methods of the present invention can be used to increase proliferation of fibroblast cells and, more particularly, in the treatment of wounds as well as strengthening of the skin.

### FIELD OF THE INVENTION

The present invention relates to methods and compositions for increasing mammalian cell proliferation.

### BACKGROUND OF THE INVENTION

Cell proliferation is involved in various biological functions. It is important for maintenance of healthy skin and plays a significant role in wound healing. Fibroblasts, endothelial cells and keratinocytes are indispensable in cutaneous wound repair. All three cell types play vital roles in the initial phase of wound healing. Fibroblasts migrate into the wound site within about 24 hours after injury. During a later phase of healing (typically about 4-21 days), fibroblasts are activated and undergo a burst of proliferative and synthetic activity. They produce a high amount of fibronectin and synthesize other proteinaceous components of extracellular matrix, including collagen, elastin and glycosaminglycans. Fibroblasts are also known to contribute in contraction of the wound (Cherry, G. W., Hughes, M. A., Ferguson, M. W. J. and Leaper D. J., Wound Healing. In Oxford Textbook of Surgery. P. J. Morris, and W. C. Wood, (eds.), pp. 131-159, Vol. 1, Oxford University Press, New York, 2001, ISBN: 0192628844). Accordingly, fibroblast proliferating agents have therefore been shown to increase the wound healing process. See, for example, S. Casadio et al. On the Healing Properties of Esters of D-panthenol with Terpene Acids, with Particular Reference to D-pantothenyl Trifarnesylacetate. Arzneimittelforschung., 17, 1122-1125, (1967); M. Aprahamian et al., Effects of Supplemental Pantothenic Acid on Wound Healing: Experimental Study in Rabbit, American Journal of Clinical Nutrition 41, 578-589 (1985), B. J. Weimann and et al., Studies on Wound Healing: Effects of Calcium D-Pantothenate on the Migration, Proliferation and Protein Synthesis of Human Dermal Fibroblasts in Culture, International Journal of Vitamin Nutrition Res., 69, 113-119, (1999).

The body produces many substances generally known as growth factors such as, for example, platelet-derived growth factor (PDGF), platelet-derived angiogenesis factor (PDAF), vascular endothelial growth factor (VEGF), platelet-derived epidermal growth factor (PDEGF), platelet factor 4 (PF4), transforming growth factor beta (TGF-B), transforming growth factor alpha (TGF-A), insulin-like growth factors 1 and 2 (IGF-1 and IGF-2), beta thromboglobulin-related proteins (BTG), thrombospondin (TSP), fibronectin, von Wallinbrand's factor (vWF), angiogenin, keratinocyte growth factor (KGF), epidermal growth factor (EGF), fibroblast growth factor (FGF), and so forth. One of the important characteristics common to each substance is that each such substance is known or believed to enhance cell or tissue growth. Other cell proliferating agents have also been reported such as, for example, those described in EP 0953354, JP 09-227413, JP 53-062815 A2 and EP 0560845 A1. However, there exists a continuing need for additional and/or improved cell proliferating agents and compounds.

The condition of the skin is always affected by factors such as humidity, ultraviolet rays, cosmetic compositions, aging, diseases, stress and eating habits. As a result, various skin troubles can arise. The skin also becomes less resilient with age as illustrated by the formation of wrinkles. Aging is generally associated with the thinning and general degradation of skin. As the skin naturally ages, there is a reduction in the number of cells and blood vessels that supply the skin. There is also a flattening of the dermal-epidermal junction that results in weaker mechanical resistance of this junction. As a consequence, older persons are more susceptive to blister formation in cases of mechanical trauma or disease processes. (Oikarinen, A., The Aging of Skin: Chronoaging Versus Photoaging, Photodermatal. Photoimmunol. Photomed., 7, 3-4, 1990).

The skin also contains an elaborate network of elastin fibers that are responsible for maintaining its elastic properties. With excessive exposure to sunlight the elastic fiber system becomes hyperplastic, disorganized and ultimately disrupted. This process is known as actinic elastosis and it is the principal cause of wrinkling, discoloration and laxity of the skin in the exposed areas of the body. As new fibroblasts, endothelial cells and keratinocytes form, the skin can repair itself. However, the skin becomes less able to do so as it ages. Therefore, agents that can accelerate the growth and repair of prematurely aged skin are needed.

Damage or injury to tissues and/or organs is a common occurrence. The body is most often able to isolate the damaged area and then repair itself by removing and replacing damaged tissue. Injury to tissues and organs can originate from a great variety of sources such as, for example, trauma, UV degradation, toxic and/or pathogenic degradation, thermal degradation (e.g., excessive heat or cold), and so forth. While the body has an impressive array of response mechanisms that limit tissue damage and promote repair, methods of increasing the speed and degree of repair are continually being sought out. In this regard, increasing the speed and/or degree that injuries are healed is beneficial in that it (i) decreases the pain and discomfort commonly associated with the wound and wound healing process; (ii) decreases the chances of developing an infection or other ailment during a period when the tissue or organ has a reduced capacity to ward off illnesses; and (iii) reduces health costs associated with treating such conditions.

In this regard, and by way of example, the skin is the largest organ in the body and not surprisingly, wounds and injuries to the skin are a common occurrence. Healing of wounds in the skin has three general phases including (1) inflammation, migration and proliferation; (2) repair which includes the formation of collagen and other compounds; (3) wound closure. Initially, inflammatory cells and other cells migrate into and fill the damaged area. Then, in the repair phase, new connective tissues are formed from fibronectin, which in turn results in the production of collagen fibrils and eventually larger collagen fibers. The wound is thereafter closed by wound contraction which results, in part, by the modified fibroblasts present in and around the wound.

Plant growth factors play an integral role in growth and development of plants. Plant hormones are major plant growth factors. They are naturally occurring organic molecules which are effective in small concentrations. Plant hormones are divided into five main classes: Auxins, Gibberellins, Cytokinins, Ethylene and Abscisic acid (Janick, J., Horticultural Science, pp. 95-121, W. H. Freeman and Company, San Francisco, 1979, ISBN: 0716710315; Biology of Plants, P. H. Raven, R. F. Evert and H. Curtis (Eds.), pp.483-496, Worth Publishers Inc., New York, 1976, ISBN: 0879010541; Biology, B. S. Guttman and J. W. Hopkins (Eds.), pp. 822-841, McGraw-Hill, New York, 1998, ISBN: 0697223663). Auxins stimulate cell elongation in shoot tips, embryos, young leaves, flowers, fruits, and pollen. Gibberellins stimulate cell division and elongation. Cytokinins stimulate mitosis in actively developing plant parts. They stimulate cell division. Ethylene, a gaseous plant hormone, speeds ripening. Abscisic acid inhibits the growth-inducing effects of other hormones.

Kinetin, a Cytokinin, is reported to delay the onset of aging characteristics in human fibroblasts (S. I. Rattan, Biochem Biophys Res. Comm., 201, 665-672, 1994). Gibberellic acid is a Gibberellin also known as Gibberellin A3. Zeatin is a Cytokinin. Jasmonic acid is a naturally occurring plant growth factor.

There is no prior art on human fibroblast cell proliferating effects of plant growth factors including gibberellic acid, kinetin, zeatin and jasmonic acid.

U.S. Pat. No. 6,174,541, issued Jan. 16, 2001 to Song et al., herein incorporated by reference, describes improved wound healing associated with a plant hormone, Indole-3-Acetic acid (an Auxin). As mentioned therein and with the exception of that reference, there has been no other reported correlation between the use of plant growth hormones and wound healing in human cell studies.

### SUMMARY OF THE INVENTION

It has been found that certain plant growth factors significantly increase cell proliferation and thus can be used to treat conditions or maladies where increased cell growth would be beneficial. In one aspect, a cell proliferating composition is provided including a therapeutically effective amount of a plant growth factor selected from the group consisting of gibberellic acid, kinetin, zeatin and jasmonic acid and derivatives thereof. In one embodiment, the plant growth factor is present in the composition in an amount between about 0.0001 percent and about 90 percent (by weight). In a further embodiment, the plant growth factor is present in the composition in an amount between about 0.01 percent to 5 percent (by weight). In yet another embodiment, the cell proliferating composition can include one or more pharmaceutically acceptable carriers.

In an additional embodiment, a composition for treating wounds is provided including (i) an effective amount of a plant growth factor selected from the group consisting of gibberellic acid, kinetin, zeatin and jasmonic acid, and derivatives thereof; and (ii) a pharmaceutically acceptable carrier. In one aspect the plant growth factor is present in an amount between about 0.0001 percent and about 90 percent (by weight) of the composition. In a further embodiment, the plant growth factor is present in the composition in an amount between about 0.01 percent to 5 percent (by weight). In a further aspect, the plant growth factor is present in an amount sufficient to increase fibroblast cell growth at least 2 percent. In one embodiment, the pharmaceutical carrier is selected from the group consisting of ointments, creams, gels, foams, sprays, salves, films, and fabrics. In a further embodiment, the composition is a semi-solid material and includes a base selected from the group consisting of hydrocarbon bases, absorption bases, water-removable bases and water-soluble bases. In still a further embodiment, the composition can include one or more active agents selected from the group consisting of emollients, anti-infective agents, preservatives, pH modifiers, mechanical protectants, chemical protectants, adsorbents and humectants.

In a further aspect, methods of treating wounds, increasing cell proliferation and of promoting healthy skin development are provided including the steps of applying to and/or treating tissue containing fibroblast cells with one of the pharmaceutical compositions described herein.

### BRIEF DESCRIPTION OF THE DRAWINGS

FIG. 1 is a bar graph illustrating the effect of gibberellic acid on cell proliferation.

FIG. 2 is a bar graph illustrating the effect of kinetin on cell proliferation.

FIG. 3 is a bar graph illustrating the effect of zeatin on cell proliferation.

FIG. 4 is a bar graph illustrating the effect of jasmonic acid on cell proliferation.

### DESCRIPTION OF THE INVENTION

Reference will now be made in detail to embodiments of the present invention, various specific examples of which will be discussed herein. Each embodiment is provided by way of explanation of the invention, and not meant as a limitation of the invention. For example, features illustrated or described as part of one embodiment may be used with another embodiment to yield still further embodiments. It is intended that the present invention include these and other modifications and variations as come within the spirit of the invention. In addition, throughout the disclosure various theories or mechanisms relating to the present invention are provided. However, the inventor does not wish to be bound by the same and the theories or mechanisms are provided solely to better understand the present invention and are not intended to limit the effective scope of the claims. Further, as used herein, the term “comprising” is inclusive or open-ended and does not exclude additional unrecited elements, compositional components, or method steps. Accordingly, the term “comprising” encompasses the more restrictive terms “consisting essentially of” and “consisting of.”

As indicated above, certain plant growth factors have been found to have a cell proliferating effect on mammalian cells and, in particular, upon the proliferation of connective tissue cells such as, for example, fibroblasts. Desirably, the cells and conditions to be treated with the therapeutic compositions and methods of the present invention are those of mammals. Mammals include various classes and families of animals including, but not limited to, primates, bovines, canines, equines, felines, etc. As specific examples, mammals include humans, certain farm animals (e.g., cattle, horses, pigs, etc.), certain lab animals (e.g., mice, rats, rabbits, etc.), many pets and zoo animals (e.g., dogs, cats, monkeys, etc.).

The compositions and methods described herein are believed generally suitable for use in treating conditions where cell proliferation is desirable. By way of non-limiting example, increased proliferation of fibroblasts would be highly beneficial in the treatment of wounds. As a further example, increased proliferation of fibroblast cells would be beneficial in the treatment of the skin by improving the extra-cellular matrix thereby tightening and/or strengthening the skin. More particularly, collagen, the predominant matrix skin protein, is known to impart tensile strength to skin. It has been shown that collagen is significantly reduced with age and UV exposure. The degradation or destruction of the architecture of these proteins decreases the tensile strength of the skin causing wrinkles and laxity. Many studies involving human subjects have shown that collagen type I is decreased with increasing severity of photodamage. See, for example, A. Kligman, Early Destructive Effect Of Sunlight On Human Skin, JAMA, 210, 2377-2380 (1969); R. Lavker, Structural Alterations In Exposed And Unexposed Aged Skin, Journal of Inv. Derm. 73, 59-66 (1979); J. Smith et al., Journal of Investigative Dermatology, 39, 347-350 (1962); and S. Shuster et al., The Influence of Age And Sex On Skin Thickness, Skin Collagen And Density, British Journal of Dermatology, 93, 639-643 (1975). In addition, some correlation in the histology of wrinkles and reduction in collagen levels in the sun-exposed skin has been reported. See, for example, S. Chen et al., Effects of all-Trans Retinoic Acid on UVB-Irradiated and Non-Irradiated Hairless Mouse Skin, Journal of Investigative Dermatology 98, 248-254 (1992). The restoration of collagen type I in photodamaged human skin by a topical treatment has also been reported. See, for example, C. Griffiths, et al., Restoration of Collagen Formation in Photodamaged Human Skin by Tretinoin (Retinoic Acid). The New England Journal of Medicine, 329, 530-535 (1993). Thus, it is believed that the cell proliferating compositions and methods of the present invention would also be beneficial to the repair and/or prevention of cutaneous tissue damage associated with exposure to the elements as well as aging due to time itself due to the ability to increase the number of fibroblasts.

As indicated above, the present invention also provides methods and therapeutically effective compositions for treating wounds. The wounds can be external or internal and as used herein the term “wound” includes tissue that has been incised, lacerated, perforated, abraded, burnt or otherwise degraded. Within the larger class of wounds are acute wounds, chronic wounds, minor cuts and burns. As used herein, the term “acute wound” means when the skin is injured as a result of traumatic abrasion, laceration or superficial damage and heals spontaneously without complications through normal phases of wound healing (hemostasis, inflammation, proliferation and remodeling).

As used herein, the term “chronic wound” means that the body's natural healing process is delayed due to an underlying pathologic process for example vascular insufficiency. Unlike acute wounds, there is no clot formation in chronic wounds and they normally occur in compromised patients who are less able to heal.

As a particular example, the therapeutic compositions and methods of the present invention can be used to promote the healing of wounds in cutaneous and/or subcutaneous tissues and also to regenate tissue in damaged organs. Epidermal, dermal and underlying subcutaneous tissues as well as organs suffer from various wounds and healing can be improved in any or all of these tissues utilizing the therapeutic compositions and methods of the present invention. In addition, the therapeutic compositions and methods of the present invention can be used to promote healthy skin development.

The increased proliferation of fibroblasts, endothelial cells and/or keratinocytes increases the availability of fibronectin and other proteinaceous components which are necessary for the production of collagen, elastin and glycosaminglycans. In addition, it is noted that collagen is a major component of connective tissue matrices, not only in skin, but also in other tissues such as, for example, lungs, bone, synovium, eye, tendons, cartilage and gingiva. In this regard, there is a high correlation between proliferation of fibroblasts and tissue healing.

Thus, while the invention is often described with relation to wound healing and/or general strengthening of cutaneous and subcutaneous tissue, comparable beneficial cell proliferation effects would be expected in other cells and tissues and in particular those containing fibroblasts and/or collagen. Therefore, the therapeutic compositions and methods of the present invention are believed useful in treating any traumatized or degraded body tissue in which the increased growth of fibroblasts, keratinocytes, epithelial cells or similar cells is beneficial to or otherwise improves healing and/or maintenance of the tissue.

### Plant Growth Factors

As indicated above, the present invention relates to the use of therapeutically effective compositions and methods comprising certain plant growth factors in order to increase cell proliferation and thereby treat and/or prevent various maladies. Plants produce many substances generally known as growth factors such as, for example, hormones, jasmonates, brassinosteroids, salicylates, systemin and polyamines.

There are five major types of plant hormones: Auxins, Gibberellins, Cytokinins, Ethylene and Abscisic acid. The roles of each of these in plant growth have been described above. Three plant hormones pertinent to the present invention are gibberellic acid (a Gibberellin), kinetin (a Cytokinin), and zeatin (a Cytokinin). Other types of plant growth factors include jasmonates, brassinosteroids, salicylates, systemin and polyamines.

Gibberellic acid has the following formula: C₁₉H₂₂O₆. Kinetin has the following formula: C₁₀H₉N₅O. Zeatin has the following formula: C₁₀H₁₃N₅O. Jasmonic acid, as mentioned above, is a naturally occurring plant growth factor having the following formula: C₁₂H₁₈O₃. Jasmonic acid is involved in the plant wound response and defense mechanism.

It has been found that certain plant growth factors can be used to increase mammalian cell proliferation. In a particular embodiment, the plant growth factors are utilized in a therapeutically effective amount to improve healing in tissues such as cutaneous tissue, other tissue and to improve the skin. As used herein an “effective amount” or a “therapeutically effective amount” refers to an amount that is sufficient to increase cell proliferation. In this regard, increased cell proliferation is relative to normal cell growth rates for like tissue, i.e., similar in age, nature or degree of damage, etc. In a particular embodiment, the desired tissues and/or cells are treated with one or more of the aforesaid plant growth factors in an amount sufficient to increase cell growth rate by more than 1 percent. In a particular embodiment, cutaneous, subcutaneous and/or other tissues are treated with one or more of the aforesaid plant growth factors in an amount sufficient to increase fibroblast growth rates at least 2 percent and, still more desirably, in an amount sufficient to increase fibroblast growth rate at least about 5 percent and, even still more desirably, in an amount sufficient to increase fibroblast growth rate at least about 10 percent.

### Pharmaceutical Preparations and Compositions

The therapeutically effective compositions of the present invention can be administered by various methods including systemically, orally, topically, intravenously, intramuscularly, transdermally, transnasally, transmucosally, rectally and/or locally. The therapeutically effective compositions may be stored for future use or may be formulated in effective amounts within pharmaceutically acceptable carriers to prepare a wide variety of pharmaceutical compositions. Examples of pharmaceutically acceptable carriers are pharmaceutical appliances, topical vehicles (non-oral and oral), ingestible vehicles and so forth. In addition, the pharmaceutical compositions of the present invention can be made using manufacturing techniques and processes readily known to those skilled in the art.

Examples of pharmaceutical appliances are sutures, staples, gauze, bandages, burn dressings, artificial skins, liposome or micell formulations, microcapsules, aqueous articles for soaking gauze dressings, and so forth. In addition, ingestible compositions desirably can employ ingestible or partly ingestible vehicles such as confectionary bulking agents which include hard and soft vehicles such as, for example, tablets, suspensions, chewable candies or gums, lozenges and so forth.

Topical compositions may employ one or more carriers or vehicles such as, for example, creams, gels, foams, ointments, sprays, salves, bio-adhesives, films, fabrics and so forth, which are intended to be applied to the skin or a body cavity. Topical compositions may also be adapted for use as an oral vehicle such as, for example, mouthwashes, rinses, oral sprays, suspensions, and dental gels, which are intended to be taken by mouth but are not intended to be ingested. Topical ointments and other semi-solid compositions commonly employ one or more bases as a vehicle for drug delivery. Exemplary bases include, but are not limited to, hydrocarbon bases (e.g., white petrolatum, white ointment, vegetable oils, animal fats, etc.), absorption bases (e.g., hydrophilic petrolatum, anhydrous lanolin, lanolin, cold cream, etc.), water-removable bases (e.g., hydrophilic ointment USP, ethoxylated fatty alcohol ethers, ethoxylated lanolin derivatives, sorbitan fatty acid esters, etc.), and water-soluble bases (e.g., polyethylene glycol ointment, etc.). As further specific examples thereof, topical compositions believed suitable for use with the invention are described in U.S. Pat. No. 6,046,160, the entire contents of which are incorporated herein by reference.

A variety of traditional ingredients may optionally be included in the pharmaceutical compositions in effective amounts. By way of non-limiting example, the pharmaceutical compositions can contain one or more of the following materials: fillers, diluents, cleaning agents, buffers, preservatives, pH and toxicity modifiers, mechanical protectants, chemical protectants, adsorbents, antioxidants, viscosity modifiers, extenders, excipients, astringents, emollients, demulcents, humectants, emulsifiers, transdermal delivery enhancing agents, controlled-release agents, dyes or colorants, stabilizers, lubricants and so forth. These and other conventional pharmaceutical additives known to those having ordinary skill in the pharmaceutical arts can be used in the pharmaceutical composition as dictated by the nature of the delivery vehicle.

The amounts of additional components within the compositions are readily determined by those skilled in the art without the need for undue experimentation and will vary with the nature of the vehicle (e.g., gel versus a spray), the wound to be treated, frequency of treatment and so forth. Thus, the amount of therapeutic wound healing composition may be varied in order to obtain the result desired in the final product and such variations are within the capabilities of those skilled in the art without the need for undue experimentation. In a particular embodiment, the pharmaceutical composition can comprise a pharmaceutical composition having one or more plant growth factors present in an amount less than 90 percent by weight of the pharmaceutical composition and in a further embodiment in an amount less than about 20 percent by weight of the pharmaceutical composition. In a further embodiment, the pharmaceutical compositions can contain one or more of the aforesaid plant growth factors in an amount between about 0.0001 percent to about 90 percent, by weight of the pharmaceutical composition. In an alternative embodiment, the pharmaceutical composition comprises one or more of the aforesaid plant growth factors in an amount between about 0.01 percent to about 5 percent by weight of the pharmaceutical composition.

### EXAMPLES

The proliferative response of plant growth factors on the human skin fibroblast cell line (Clonetics, Walkersville, Md., normal human dermal fibroblasts, neonatal, Catalog No. CC-2509) was determined in a 96-well assay system using serum-free medium as a control.

### Example 1

Stock solution of gibberellic acid (Sigma Chemical Company, St. Louis, Mo.) (0.001 M) was prepared in water and then diluted with serum-free Dulbecco's Modified Eagle's Medium (DMEM, Sigma Chemical Co., St. Louis, Mo.) to 10⁻⁵, (“GA2”) and 10⁻⁶ (“GA1”) M solutions. Cells were seeded into 96 well plates at a concentration of 3×10³ cells in 100 microliters of DMEM containing 10 percent fetal bovine serum (FBS, Sigma Chemical Co., St. Louis, Mo.). Plates were incubated for 24 hours at 37° C. in a humidified, 5 percent CO₂ atmosphere. After incubation, the medium was aspirated and the wells were rinsed twice with 100 microliters of serum-free DMEM. The final rinse was aspirated and 100 microliters of the 10⁻⁵ and 10⁻⁶ M of each solution was added to 10 wells. In addition, 100 microliters of vehicle (serum-free DMEM) was added to 10 wells as control. All wells were incubated for 28 hours at 37° C. in a humidified, 5 percent CO₂ atmosphere. After incubation, 20 microliters of Cell Titer 96 Aqueous One Solution Reagent (Promega, Corp., Madison, Wis.) was added to all wells. The plates were swirled gently and placed back in the incubator for 45 minutes and spectrophotometric absorbance was read at 490 nm.

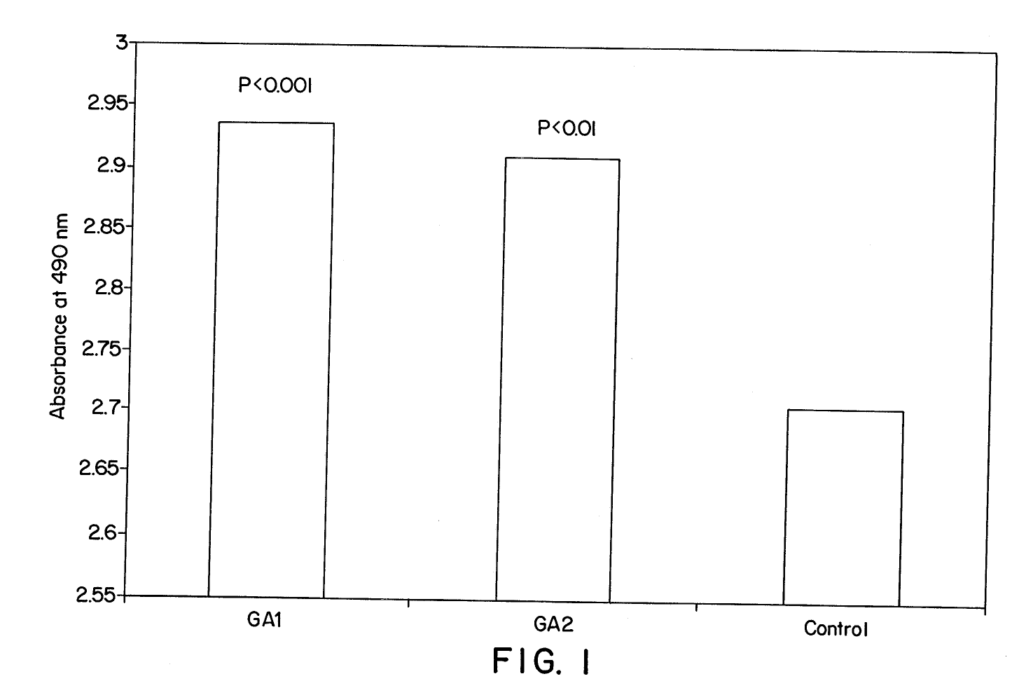

Statistical analysis was done by using one-way ANOVA. A statistically significant difference was observed between the control and gibberellic acid. Based on significant statistical differences, gibberellic acid appears to be a good cell proliferating agent (FIG. 1).

### Example 2

Kinetin (Spectrum Chemical, CA) 1 mg/ml aqueous solution as supplied by the Company was used as a stock solution. Further dilutions were made with serum-free Dulbecco's Modified Eagle's Medium (DMEM, Sigma Chemical Co., St. Louis, Mo.) to 10⁻⁵, (“K2”) and 10⁻⁶ (“K1”) M solutions. Cells were seeded into 96 well plates at a concentration of 3×10³ cells in 100 microliters of DMEM containing 10 percent fetal bovine serum (FBS, Sigma Chemical Co., St. Louis, Mo.). Plates were incubated for 24 hours at 37° C. in a humidified, 5 percent CO₂ atmosphere. After incubation, the medium was aspirated and the wells were rinsed twice with 100 microliters of serum-free DMEM. The final rinse was aspirated and 100 microliters of the 10⁻⁵ and 10⁻⁶ M of each solution was added to 10 wells. In addition, 100 microliters of vehicle (serum-free DMEM) was added to 10 wells as control. All wells were incubated for 28 hours at 37° C. in a humidified, 5 percent CO₂ atmosphere. After incubation, 20 microliters of Cell Titer 96 Aqueous One Solution Reagent (Promega, Corp., Madison, Wis.) was added to all wells. The plates were swirled gently and placed back in the incubator for 45 minutes and spectrophotometric absorbance was read at 490 nm.

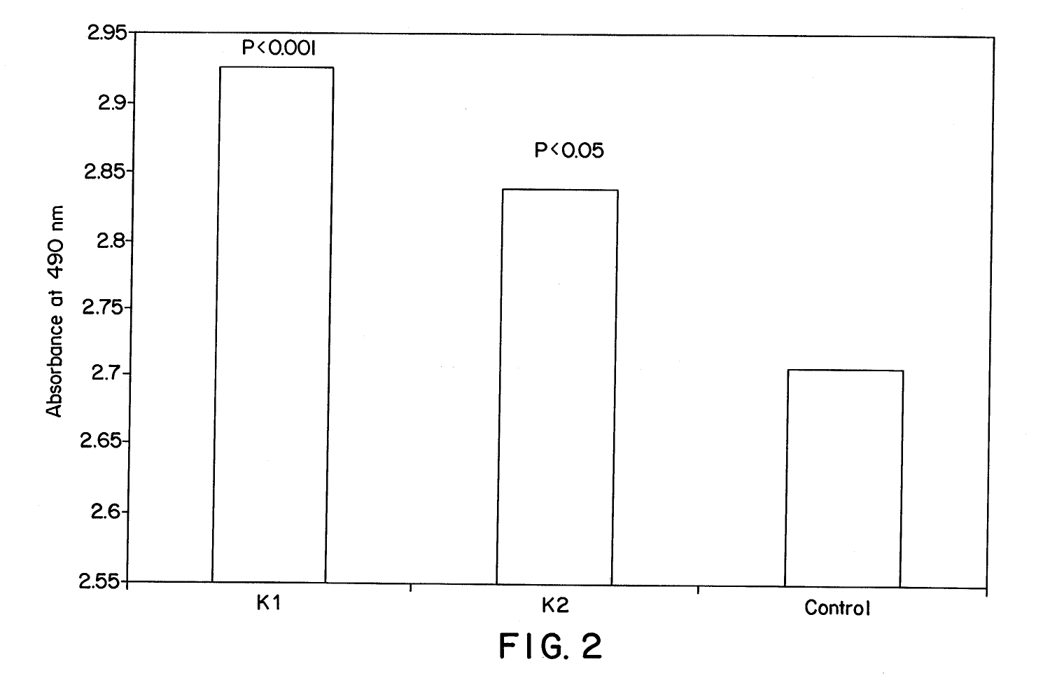

Statistical analysis was done by using one-way ANOVA. A statistically significant difference was observed between the control and kinetin. Based on significant statistical differences, kinetin appears to be a good cell proliferating agent (FIG. 2).

### Example 3

Stock solution of trans-zeatin HCl (Sigma Chemical Company, St. Louis, Mo.) (0.001 M) was prepared in water and then diluted with serum-free Dulbecco's Modified Eagle's Medium (DMEM, Sigma Chemical Co., St. Louis, 2 Missouri) to 10⁻⁴ (“Z3”), 10⁻⁵ (“Z2”), and 10⁻⁶ (“Z1”) M solutions. Cells were seeded into 96 well plates at a concentration of 2×10³ cells in 100 microliters of DMEM containing 10 percent fetal bovine serum (FBS, Sigma Chemical Co., St. Louis, Mo.). Plates were incubated for 24 hours at 37° C. in a humidified, 5 percent CO₂ atmosphere. After incubation, the medium was aspirated and the wells were rinsed twice with 100 microliters of serum-free DMEM. The final rinse was aspirated and 100 microliters of the 10⁻⁴-10⁻⁶ M of each solution was added to 20 wells. In addition, 100 microliters of vehicle (serum free DMEM) was added to 10 wells as control. All wells were incubated for 28 hours at 37° C. in a humidified, 5 percent CO₂ atmosphere. After incubation, 20 microliters of Cell Titer 96 Aqueous One Solution Reagent (Promega, Corp., Madison, Wis.) was added to all wells. The plates were swirled gently and placed back in the incubator for 45 minutes and spectrophotometric absorbance was read at 490 nm.

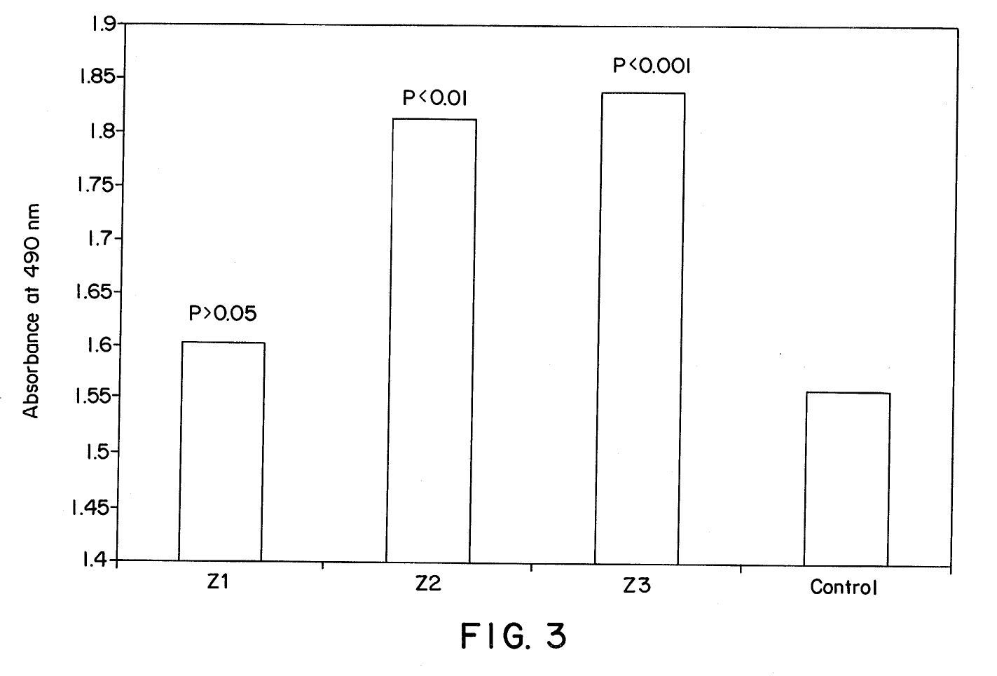

Statistical analysis was done by using one-way ANOVA. A statistically significant difference was observed between the control and trans-zeatin. Based on significant statistical differences, zeatin appears to be a good cell proliferating agent (FIG. 3).

### Example 4

Stock solution of jasmonic acid (Sigma Chemical Company, St. Louis, Mo.) (0.238 M) was prepared in ethanol and then diluted with serum-free Dulbecco's Modified Eagle's Medium (DMEM, Sigma Chemical Co., St. Louis, Mo.) to 10⁻⁴ (“JA3”), 10⁻⁵ (“JA2”), and 10⁻⁶ (“JA1”) M solutions. Cells were seeded into 96 well plates at a concentration of 2×10³ cells in 100 microliters of DMEM containing 10 percent fetal bovine serum (FBS, Sigma Chemical Co., St. Louis, Mo.). Plates were incubated for 24 hours at 37° C. in a humidified, 5 percent CO₂ atmosphere. After incubation, the medium was aspirated and the wells were rinsed twice with 100 microliters of serum-free DMEM. The final rinse was aspirated and 100 microliters of the 10⁻⁴-10⁻⁶ M of each solution was added to 20 wells. In addition, 100 microliters of vehicle (serum free DMEM) was added to 10 wells as control. All wells were incubated for 28 hours at 37° C. in a humidified, 5 percent CO₂ atmosphere. After incubation, 20 microliters of Cell Titer 96 Aqueous One Solution Reagent (Promega, Corp., Madison, Wis.) was added to all wells. The plates were swirled gently and placed back in the incubator for 45 minutes and spectrophotometric absorbance was read at 490 nm.

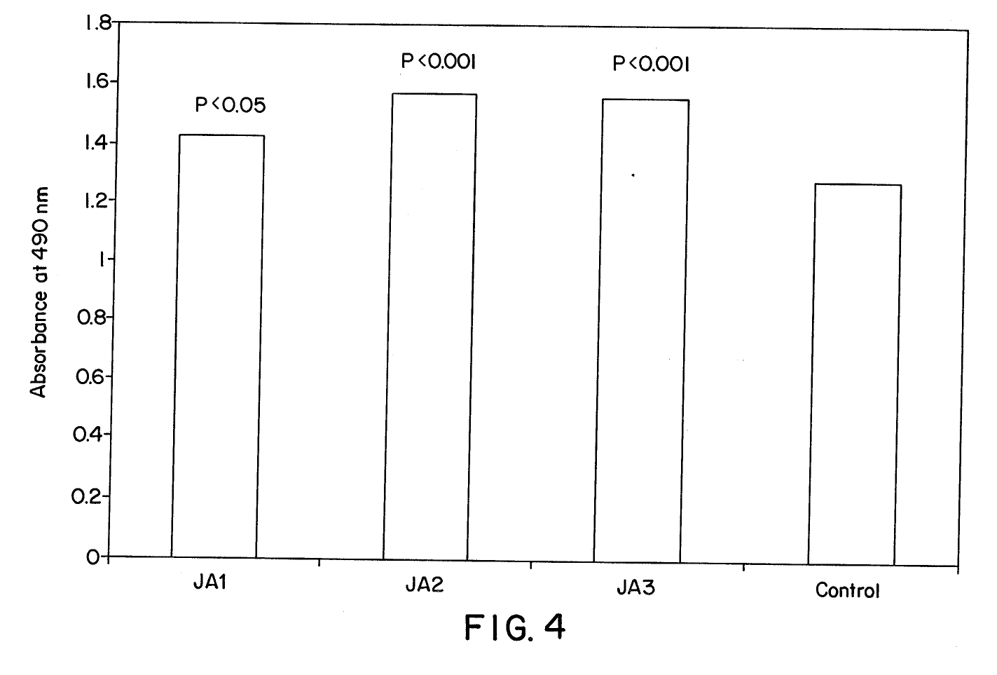

Statistical analysis was done by using one-way ANOVA. A statistically significant difference was observed between control and jasmonic acid. Based on significant statistical differences, jasmonic acid appears to be a strong cell proliferating agent (FIG. 4).

Cell growth rates for other cell lines may be determined in a similar manner. However, one skilled in the art will appreciate that various aspects of the test will change in accord with the particular cell line being evaluated.

We claim:

1. A cell proliferating composition comprising a therapeutically effective amount of a plant growth factor selected from the group consisting of gibberellic acid, kinetin, zeatin, jasmonic acid and derivatives thereof.

2. The cell proliferating composition of claim 1 wherein said plant growth factor is present in an amount between about 0.0001 percent and about 90 percent by weight.

3. The cell proliferating composition of claim 1 wherein said plant growth factor is present in an amount between about 0.01 percent and about 5 percent by weight.

4. The cell proliferating composition of claim 1 wherein said plant growth factor comprises gibberellic acid.

5. The cell proliferating composition of claim 1 wherein said plant growth factor comprises kinetin.

6. The cell proliferating composition of claim 1 wherein said plant growth factor comprises zeatin.

7. The cell proliferating composition of claim 1 wherein said plant growth factor comprises jasmonic acid.

8. The cell proliferating composition of claim 1 further including a pharmaceutical carrier.

9. A composition for treating wounds comprising:
a) an effective amount of a plant growth factor selected from the group consisting of gibberellic acid, kinetin, zeatin, jasmonic acid and derivatives thereof; and
b) a pharmaceutical carrier.

10. The composition of claim 9 wherein said plant growth factor is present in an amount between about 0.0001 percent and about 90 percent.

11. The composition of claim 9 wherein said plant growth factor is present in an amount between about 0.01 percent and about 5 percent by weight.

12. The composition of claim 9 wherein said plant growth factor comprises gibberellic acid.

13. The composition of claim 9 wherein said plant growth factor comprises kinetin.

14. The composition of claim 9 wherein said plant growth factor comprises zeatin.

15. The composition of claim 9 wherein said plant growth factor comprises jasmonic acid.

16. The composition of claim 9 wherein said carrier is selected from the group consisting of ointments, creams, gels, foams, sprays, salves, films, and fabrics.

17. The composition of claim 9 wherein said composition is a semi-solid material and includes a base selected from the group consisting of hydrocarbon bases, absorption bases, water-removable bases and water-soluble bases.

18. The composition of claim 17 further comprising at least one active agent selected from the group consisting of emollients, anti-infective agents, preservatives, pH modifiers, mechanical protectants, chemical protectants, adsorbents, and humectants.

19. A method of increasing cell proliferation comprising treating a tissue with a therapeutically effective amount of a plant growth factor selected from the group consisting of gibberellic acid, kinetin, zeatin, jasmonic acid and derivatives thereof.

20. The method of claim 19 comprising treating the tissue with a pharmaceutical composition containing said plant growth factor and wherein said pharmaceutical composition includes less than about 20 percent by weight of said plant growth factor.

21. The method of claim 19 wherein said plant growth factor comprises gibberellic acid.

22. The method of claim 19 wherein said plant growth factor comprises kinetin.

23. The method of claim 19 wherein said plant growth factor comprises zeatin.

24. The method of claim 19 wherein said plant growth factor comprises jasmonic acid.

25. The method of claim 19 wherein said plant growth factor is administered by a method selected from the group consisting of orally, topically, intravenously, intramuscularly, transdermally, transnasally, transmucosally and rectally.

26. The method of claim 19 wherein said tissue comprises cutaneous tissue.

27. The method of claim 26 wherein said plant growth factor is treated by topically applying said therapeutically effective amount of plant growth factor.

28. A method of treating a wound comprising:
a) providing a pharmaceutical composition containing a therapeutically effective amount of a plant growth factor selected from the group consisting of gibberellic acid, kinetin, zeatin, jasmonic acid and derivatives thereof; and
b) treating the wound with said pharmaceutical composition.

29. The method of claim 28 wherein said wound comprises an acute wound.

30. The method of claim 28 wherein said wound comprises a chronic wound.

31. The method of claim 28 wherein said wound comprises a burn.

32. The method of claim 28 wherein said plant growth factor comprises between about 0.0001 percent and about 90 percent by weight of said pharmaceutical composition.

33. A method of promoting healthy skin development comprising administering a therapeutically effective amount of a plant growth factor selected from the group consisting of gibberellic acid, kinetin, zeatin, jasmonic acid and derivatives thereof.
